(19) United States
(12) Patent Application Publication
(10) Pub. No.: US 2025/0204856 A1
(43) Pub. Date: Jun. 26, 2025

(54) FLEXIBLE MICRO-NEEDLE ELECTRODE FOR BIOPOTENTIAL MONITORING, A METHOD FOR CONSTRUCTING THE FLEXIBLE MICRO-NEEDLE ELECTRODE AND A PATCH ELECTRODE COMPRISING THE FLEXIBLE MICRO-NEEDLE ELECTRODE

(71) Applicant: The University of Hong Kong, Pokfulam (CN)

(72) Inventors: Wendi Li, Kowloon (HK); Chuying Sun, North Point (HK); Huahui Liang, Central and Western District (HK); Liyang Chen, Pokfulam (HK)

(21) Appl. No.: 18/969,502

(22) Filed: Dec. 5, 2024

Related U.S. Application Data
(60) Provisional application No. 63/613,522, filed on Dec. 21, 2023.

(51) Int. Cl. A61B 5/00; A61B 5/262

(52) U.S. Cl. CPC A61B 5/685; A61B 5/262; A61B 5/263; A61B 5/28; A61B 5/296; A61B 2562/046; A61B 2562/125; A61B 2562/164

(57) ABSTRACT

A flexible micro-needle electrode for biopotential monitoring, a method for constructing the flexible micro-needle electrode and a patch electrode comprising the flexible micro-needle electrode. The method comprises the steps of providing a negative stamp that has been structured with a plurality of micro-needle structures; depositing at least one layer of electrically conductive material onto the negative stamp; and peeling off the at least one layer of electrically conductive material from the negative stamp to obtain the flexible micro-needle electrode comprising the at least one layer of electrically conductive material defined with the plurality of micro-needle structures.

### FIELD OF THE INVENTION

The present invention relates to a flexible micro-needle electrode for biopotential monitoring, a method for constructing the flexible micro-needle electrode and a patch electrode comprising the flexible micro-needle electrode, and, more particularly although not exclusively, to ultrathin, flexible microneedle electrodes for accurate and long-term biopotential monitoring.

### BACKGROUND OF THE INVENTION

The escalating demand for wearable electrodes in the era of digital healthcare underscores the urgent need for more efficient and accurate biopotential monitoring techniques. Surface biopotentials are crucial for assessing physical function and diagnosing diseases, which are widely used in clinical practices like electrocardiogramalectromyography (EMG), electroencephalography (EEG), and electrooculography (EOG). With advances in bioelectric acquisition technology and artificial intelligence (AI) algorithms, these biopotentials are also increasingly used in various applications such as athletic training, prosthetic control, human-robot interaction and virtual and augmented reality. The electrodes that are worn on the skin for the conversion of the body's ionic current into an electronic current measured by external electronic system play a critical role in biopotential detection. However, the current electrodes in the market often require skin pre-treatment, can cause discomfort and skin irritation, face challenges with signal degradation over time due to gel evaporation, and present limitations in terms of material cost, adhesiveness, flexibility, and scalability of production.

There has been an increasing trend toward the development of epidermal electronics. These electronic devices, such as ultra-thin and flexible electronic systems that adhere to the skin, have a wide range of applications including monitoring body conditions, providing therapeutic treatments for healthcare, drug delivery, athletic training, and human-machine interaction. As an advanced detection technology, electrophysiology has emerged as a powerful platform and is frequently adopted in epidermal electronics. Electrophysiology, including EMG, ECG, electrooculogram (EOG), and electroencephalogram (EEG), relates to biopotential signals detected at the human surface, providing information to assess the health status and diagnose abnormalities of different parts of the organism. The detection of biopotentials may rely on epidermal electrodes worn on the skin to convert the ionic current into an electrical one that can be measured by an external electronic system. Therefore, the capacity for precise, imperceptible, stable, and biocompatible long-term monitoring of electrophysiological signals represents the essential prerequisite for wearable electronics.

### SUMMARY OF THE INVENTION

In accordance with a first aspect of the present invention, there is provided a method for constructing a flexible micro-needle electrode (MNE) for biopotential monitoring comprising the steps of: providing a negative stamp that has been structured with a plurality of micro-needle structures; depositing at least one layer of electrically conductive material onto the negative stamp; and peeling off the at least one layer of electrically conductive material from the negative stamp to obtain the flexible micro-needle electrode comprising the at least one layer of electrically conductive material defined with the plurality of micro-needle structures.

In accordance with the first aspect, the negative stamp comprises a stamp substrate fabricated using nanoimprinting lithography.

In accordance with the first aspect, the negative stamp is defined with the plurality of micro-needle structures having a pyramid shape, a cone shape or a cylinder shape.

In accordance with the first aspect, the plurality of micro-needle structures include a height in a range from 20 to 200 μm, a pitch in a range from 50 to 500 μm, and a length in a range from 20 to 300 μm.

In accordance with the first aspect, the step of providing the negative stamp that has been structured with a plurality of micro-needle structures comprises the steps of providing a molding material to replicate the plurality of micro-needle structures from a positive mold; curing the molding material to provide the stamp substrate.

In accordance with the first aspect, the positive mold includes a positive PDMS mold defined with the plurality of micro-needle structures.

In accordance with the first aspect, the molding material includes a polymer molding material.

In accordance with the first aspect, the molding material is UV-curable.

In accordance with the first aspect, the negative stamp is electrically conductive, and wherein the at least one layer of electrically conductive material is deposited onto the negative stamp by electrodepositing.

In accordance with the first aspect, the negative stamp includes a layer of indium tin oxide (ITO) covering the stamp substrate.

In accordance with the first aspect, the step of providing the negative stamp that has been structured with the plurality of micro-needle structures further comprises the step of: depositing a layer of ITO onto the stamp substrate to provide the negative stamp.

In accordance with the first aspect, the layer of ITO is deposited on the stamp substrate by sputtering.

In accordance with the first aspect, the layer of ITO is approximately 250 nm thick.

In accordance with the first aspect, the at least one layer of electrically conductive material includes gold (Au) and nickel (Ni).

In accordance with the first aspect, a layer of gold and a layer of nickel are sequentially electrodeposited onto the negative stamp employing a step-up current source.

In accordance with the first aspect, the layer of gold and the layer of nickel include respectively a thickness of 500 nm and 5 μm.

In accordance with a second aspect of the present invention, there is provided a flexible micro-needle electrode (MNE) for biopotential monitoring, comprising at least one layer of electrically conductive material defined with the plurality of micro-needle structures produced using the method in accordance with the first aspect.

In accordance with a third aspect of the present invention, there is provided a patch electrode for biopotential monitoring, comprising a flexible micro-needle electrode (MNE) for biopotential monitoring in accordance with the second aspect; and an electrical conductor arranged to electrically connect the flexible micro-needle electrode to a biopotential monitoring device.

In accordance with the third aspect, the patch electrode is a dry electrode adapted to be worn by a patient.

In accordance with the third aspect, the flexible micro-needle electrode is adapted to be worn for at least twenty-four hours without loss of performance.

The invention offers a solution that features improved user comfort, reduced skin irritation, higher signal-to-noise ratio particularly in motion and more affordable unit price with an effective manufacturing process.

The present invention provides a compelling and facile approach to the fabrication of highly conductive, flexible and ultra-thin microneedle electrodes (MNEs) for accurate and imperceptible biopotential monitoring, leveraging a unique micro/nano-electroforming technique. To achieve a cost-effective and scalable fabrication process, metallic layers are electrodeposited on an Indium Tin Oxide (ITO) substrate with microneedle structures, and thus the electrodeposited metal thin film exhibits the same microstructures following the underlying ITO substrate. This approach uses the magnetron sputtering of Indium Tin Oxide (ITO) on a microneedle mold and subsequent electrodeposition of metal layers to produce ultra-thin, flexible, and highly conductive MNEs. These MNEs outperformed the electroplated planar electrodes and wet silver/silver-chloride (Ag/AgCl) electrodes.

Advantageously, the microstructures increase contact area and bypass sweat and grease on the uneven skin surface, leading to lower electrode-skin interface impedance (EII). This innovative design enhances the signal-to-noise ratio (SNR), leading to more accurate and non-invasive detection of electrophysiological signals, such as electromyograms (EMG) and electrocardiograms (ECG). In addition, the MNEs exhibited superior electro-mechanical stability, biocompatibility and comfort, making them suitable for long-term healthcare monitoring and human-robot interaction.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The foregoing and other objects and advantages of the present invention will become more apparent when considered in connection with the following detailed description and appended drawings in which like designations denote like elements in the various views, and wherein:

FIG. 1A is a process flow diagram of a method for constructing a flexible micro-needle electrode (MNE) for biopotential monitoring in accordance with an embodiment of the present invention.

FIG. 1B is an optical image of the flexible MNE fabricated in accordance with an embodiment of the present invention.

FIG. 1C is an optical microscopy image of MNE of FIG. 1B from a tilted view.

FIG. 1D is an optical microscopy image of MNE of FIG. 1B from a top view.

FIG. 2A is an illustration showing of the micro-needle structure of the MNE fabricated in accordance with an embodiment of the present invention.

FIG. 2B shows the structure on different layers of material of the microneedle array electrodes and the negative stamp being used for fabrication the MNE in the process illustrated in FIG. 1A.

FIG. 2C shows the dimensions of the structure of a microneedle array electrode of FIG. 2A.

FIG. 3A is a schematic diagram of an electrode-skin interface for wet Ag/AgCl electrodes.

FIG. 3B is an electrical equivalent circuit model of the electrode-skin interface for wet Ag/AgCl electrodes.

FIG. 3C is a schematic diagram of an electrode-skin interface for planar electrodes.

FIG. 3D is an electrical equivalent circuit model of the electrode-skin interface for planar electrodes.

FIG. 3E is a schematic diagram of an electrode-skin interface for MNE.

FIG. 3F is an electrical equivalent circuit model of the electrode-skin interface for MNE.

FIG. 4 is the equivalent circuit of an electrode-skin interface impedance (EII) test.

FIG. 5A is schematic image of a planar electrode.

FIG. 5B is schematic image of a wet Ag/AgCl electrode.

FIG. 5C is schematic image of an MNE.

FIG. 5D is an illustration of a patient wearing a pair of MNE on his arm for an EII test.

FIG. 6A is a graph depicting the EII spectra of different electrodes, covering the frequency between 1 Hz and 10⁵ Hz.

FIG. 6B is a bar graph showing the extracted EII of different electrodes at current frequencies of 1000, 100, 10, and 1 Hz.

FIG. 6C is a graph showing a comparison of EII spectra of MNEs and wet Ag/AgCl electrodes measured immediately after application and 24 hours thereafter.

FIG. 6D is a photo and a graph illustrating the normalized resistance changes of electrodes with bending radii varying from 3 to 10 mm.

FIG. 6E is a photograph of the electrode wearing positions on the skin of a patient immediately after removing the MNEs after being continuously worn for 2 hours.

FIG. 6F is a photograph of the electrode wearing positions immediately after removing the wet Ag/AgCl electrodes after being continuously worn for 2 hours.

FIG. 7A is a photograph showing skin reaction characterization at the wearing position to verify biocompatibility right after the removal of the wet Ag/AgCl electrodes and MNEs that had been continuously worn for 2 hours.

FIG. 7B is a photograph showing skin reaction characterization at the wearing position, captured 0.5 hours after the removal of the wet Ag/AgCl electrodes and MNEs that had been continuously worn for 2 hours.

FIG. 8 is a typical ECG waveform and its characteristic patterns.

FIG. 9 is a schematic illustration of ECG detection in a Lead II configuration.

FIG. 10A is a plot showing a series of ECG signals recorded by the planar electrodes.

FIG. 10B is a plot showing zoomed-in single characteristic signal recorded by the planar electrodes.

FIG. 10C is a plot showing a series of ECG signals recorded by the wet Ag/AgCl electrodes.

FIG. 10D is a plot showing zoomed-in single characteristic signal recorded by the wet Ag/AgCl electrodes.

FIG. 10E is a plot showing a series of ECG signals recorded by the MNEs.

FIG. 10F is a plot showing zoomed-in single characteristic signal recorded by the MNEs.

FIG. 11A is a plot showing simultaneous ECG signals recorded during stationary standing with planar electrodes (top), wet Ag/AgCl electrodes (middle), and MNEs (bottom).

FIG. 11B is a plot showing simultaneous ECG signals recorded during mild exercise with planar electrodes (top), wet Ag/AgCl electrodes (middle), and MNEs (bottom).

FIG. 11C is a plot showing simultaneous ECG signals recorded during vigorous exercise with planar electrodes (top), wet Ag/AgCl electrodes (middle), and MNEs (bottom).

FIG. 11D is a plot showing simultaneous ECG signals recorded during deep breathing with planar electrodes (top), wet Ag/AgCl electrodes (middle), and MNEs (bottom).

FIG. 11E is a plot showing bar graphs of the quantitative analysis of the anti-motion artifact of electrodes for the MAX-MIN of the PQRS complex.

FIG. 11F is a plot showing the standard deviation (SD) of all ECG data points.

FIG. 12A is a plot showing EMG signals captured by MNEs at gripping forces of 5 kg, 10 kg, and 15 kg.

FIG. 12B is a bar graph showing the relationship between the variations in the EMG signal amplitude and the different gripping forces.

FIG. 12C is a bar graph of the RMS signal, RMS noise, and SNR values calculated from the EMG signals during wrist lifting.

FIG. 12D is a photo demonstrating the control of a robotic hand (open hand) using EMG signals measured by the MNEs.

FIG. 12E are photos demonstrating the control of a robotic hand (close hand) using EMG signals measured by the MNEs.

FIG. 12F illustrates photos of the hand and the EMG signals generated by the flexion/extension of different fingers of the hand.

FIG. 13A shows baseline noise captured by planar electrodes.

FIG. 13B shows overall EMG signal generated by the flexor muscles under a handgrip force of 5 kg, as captured by planar electrodes.

FIG. 13C shows the EMG signals generated by the flexor muscles under a handgrip force of 5 kg, as captured by planar electrodes during muscle activation.

FIG. 13D shows baseline noise captured by the wet Ag/AgCl electrode.

FIG. 13E shows overall EMG signal generated by the flexor muscles under a handgrip force of 5 kg, as captured by the wet Ag/AgCl electrode.

FIG. 13F shows the EMG signal generated by the flexor muscles under a handgrip force of 5 kg, as captured by the wet Ag/AgCl electrode during muscle activation.

FIG. 13G shows baseline noise captured by the MNEs,

FIG. 13H shows overall EMG signals generated by the flexor muscles under a handgrip force of 5 kg, as captured by the MNEs.

FIG. 13I shows the EMG signal generated by the flexor muscles under a handgrip force of 5 kg, as captured by the MNEs during muscle activation.

FIG. 14A is an illustration showing the position of detection electrodes for EMG testing of the flexor muscles during pulling.

FIG. 14B is an illustration showing the position of detection electrodes for EMG testing of the extensor digitorum as the hand moves up and down.

FIG. 14C is an illustration showing the position of detection electrodes for EMG testing of the extensor digitorum as the hand is opened and closed.

FIG. 14D is an illustration showing the position of the detection electrodes for EMG testing of the bicep muscles as the forearm is raised and lowered.

FIG. 15A is a plot showing the SNR for flexor muscles at 5, 10 and 15 kg captured by using 3 distinct types of detection electrodes.

FIG. 15B is a plot showing the RMS Amplitude for extensor digitorum (wrist lifting) for signal, noise and SNR captured by using 3 distinct types of detection electrodes.

FIG. 15C is a plot showing the RMS Amplitude for extensor digitorum (contracting) for signal, noise and SNR captured by using 3 distinct types of detection electrodes.

FIG. 15D is a plot showing the RMS Amplitude for biceps bending the elbow to 120° for signal, noise and SNR captured by using 3 distinct types of detection electrodes.

### DETAILED DESCRIPTION OF THE INVENTION

The inventors, through their trials and experiments, devised that Ag/AgCl gel electrodes operating with an electrolytic gel may be used to obtain surface biopotentials in clinics. Despite the extensive utilization, they face several drawbacks including the necessity of skin pre-treatment, discomfort, and potential skin irritation. Moreover, they are susceptible to signal degradation during continuous monitoring due to the evaporation of the liquid in the gel electrolyte.

Alternatively, another type of electrode, dry electrodes, which do not require the utilization of electrolyte gel, could eliminate the problems associated with the wet electrodes, and thus may be an alternative for long-term biopotential detection.

Without wishing to be bound by theory, dry electrodes can further be classified into capacitive (noncontact) electrodes and dry contact electrodes. Although the capacitive electrodes exhibit better comfortability for long-time wearing without the requirement of direct contact with the skin, some may encounter significant challenges due to the ultra-high electrode-skin interface impedance (EII) and motion artifacts.

For dry electrodes, the performance may be improved by including poly (ethylenedioxythiophene):poly (styrenesulfonate) (PEDOT:PSS) layer due to its ease of processing, excellent electrical properties, transparency, and biocompatibility. Although blending it with waterborne polyurethane (WPU) and D-sorbitol, or integrating it with 2D materials like graphene, has been attempted to improve its performance, the composite material may lead to reduced conductivity (and thus performance) and complicated fabrication processes.

Preferably, noble biocompatible materials, such as gold (Au), titanium (Ti), and platinum (Pt) may be favorable for dry electrodes because of their high conductivity and good biocompatibility. However, their applications may be limited by low adhesiveness, low flexibility, and high price. More preferably, microstructured electrodes may overcome these limitations, and among various structures, electrodes with microneedles, benefiting from the microneedle structures, may exhibited lower EII due to an increased surface area and the ability to penetrate the stratum corneum, further decreasing the EII. Additionally, the microstructures may also contribute to flexibility enhancement and more conformal contact with the skin, which further improves the signal quality.

The fabrication of electrodes with microneedle structures, in some example embodiments, may heavily rely on costly processing techniques such as photolithography, etching and metal deposition. Alternative methods may rely on nanosecond laser micromachining and 3D printing, but the fabricated electrodes may exhibit a rough surface with blunt tips. Furthermore, due to the visible microneedle shape and the wearing discomfort, as well as residual traces caused by skin penetration, patients may alter their natural behavioral patterns, which can compromise the accuracy of electrophysiological quality assessments and disorder diagnosis. Collectively, these aforementioned factors may affect the performance or experiences in using transdermal microneedle electrodes.

In an example micro-molding fabrication process for manufacturing microneedle array electrodes, it involves several techniques, including standard machining, for creating microneedle templates, roll plating to prevent rust, physical and chemical reactions to form microneedle substrates, magnetron sputtering to cover the substrates with a metal layer and galvanostatic electrodeposition for surface modification. Of these techniques, machining, physical and chemical reactions, and magnetron sputtering are primarily used to produce the microneedle templates and metal layers. The fabricated microneedle arrays are comprised of PEDOT:PSS, metallic (Ti/Au) conductive layer, and the polyimide substrate.

In an alternative example, 3D printing technology may be utilized to fabricate microneedle templates. This is followed by a chemical reaction, known as the silver mirror reaction, and electroplating to construct a conductive layer composed of Silver (Ag) and Nickel (Ni). Subsequently, the 3D printed material is subjected to a chemical etching process involving a potent alkali, resulting in its erosion.

An alternative fabrication process may be used, in which the process involves several steps, including the thermal oxidation of Si sheets to form SiO₂ layers, wheel grinder cutting to create periodic grooves in a two-dimensional square column array, chemical etching of isotropic Si sheets to produce a microneedle array, erosion of SiO₂ layers using hydrofluoric acid and magnetron sputtering to deposit metal (Au or Ti) to complete the conductive layer fabrication.

In an example armband device, the armband includes an electrophysiological detection sensor that employs unstructured, dry metal electrodes, which are machined directly on a metal block.

A different type of electrode that belongs to the wet electrodes class is wet Ag/AgCl (silver/silver chloride) electrodes based on a silver/silver chloride substrate, topped with a conductive, adhesive gel. This polymer material improves adhesion to the skin, subsequently forming an equivalent circuit to gather physiological electrical signals. However, the wet electrodes have several drawbacks when compared to dry electrodes which may be further explained in this disclosure.

The present invention involves the fabrication of highly conductive, flexible, and ultra-thin microneedle electrodes (MNEs) using a templated 3D electrodeposition method. This technique is cost-effective and suitable for practical applications as it circumvents the need for expensive equipment and allows for the reuse of the electrodeposition template.

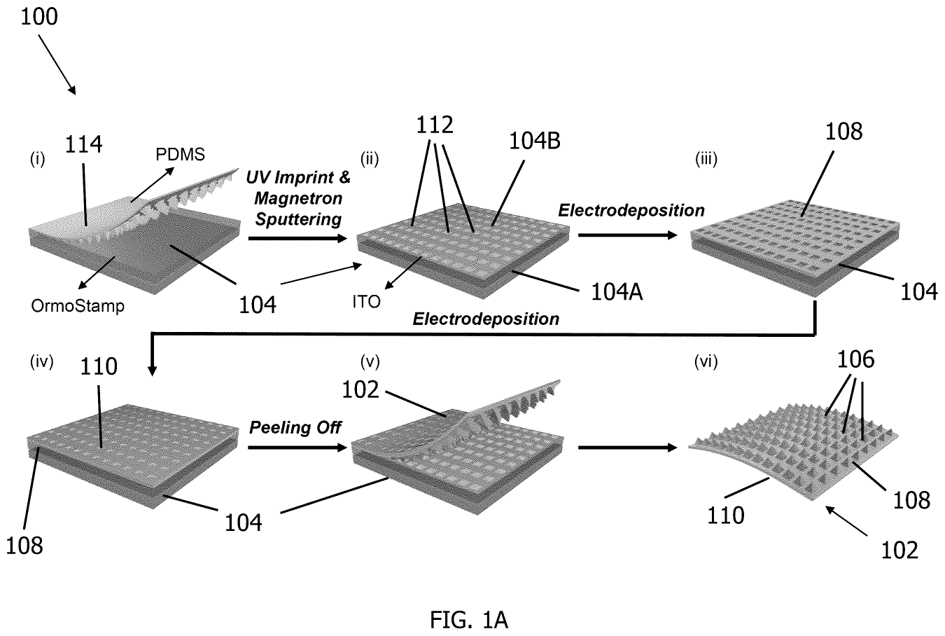

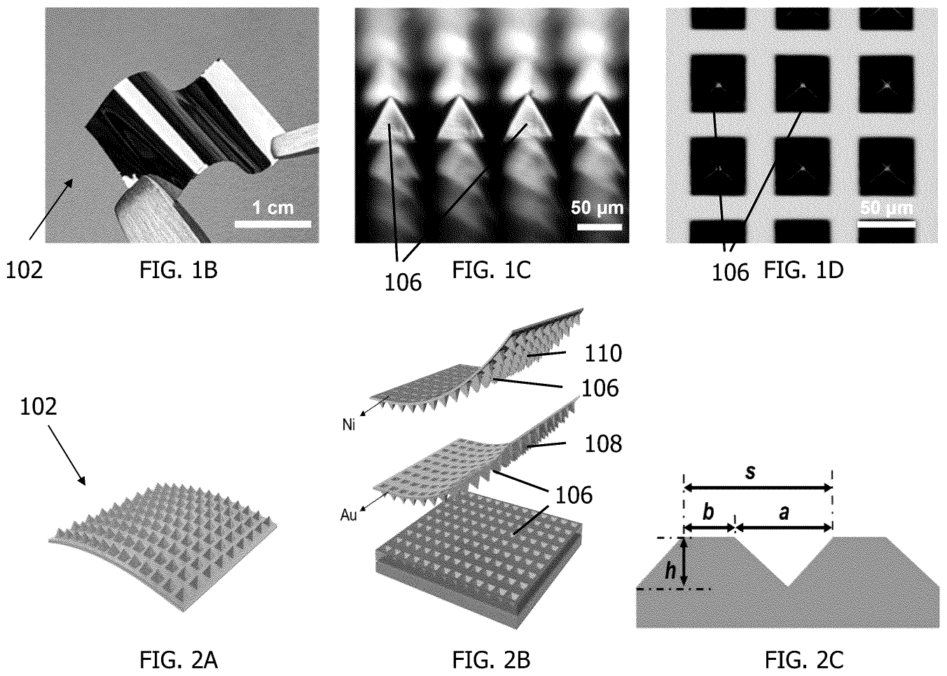

With reference to FIG. 1A, there is shown an example embodiment of a method 100 for constructing a flexible micro-needle electrode (MNE) 102 for biopotential monitoring comprising the steps of: providing a negative stamp 104 that has been structured with a plurality of micro-needle structures 106; depositing at least one layer of electrically conductive material onto the negative stamp 104; and peeling off the at least one layer of electrically conductive material from the negative stamp 104 to obtain the flexible micro-needle electrode 102 comprising the at least one layer of electrically conductive material defined with the plurality of micro-needle structures 106.

In this example, the flexible micro-needle electrode 102 may be used as an electrode for electrically connecting a biopotential monitoring device, such as monitoring devices for measuring electrocardiogram (ECG), electroencephalogram (EEG) and electromyography (EMG). The electrodes may operate as transducers, converting ionic currents from the body into electronic currents that can be measured by the device.

Referring to also to FIG. 1B to 1D, the as-fabricated micro-needle electrode 102 (i.e. the MNE in this disclosure) is substantially planar in shape, with a plurality of micro-needle structures 106 formed on one side of the planar electrode. The plurality of micro-needle structures 106 are arranged in an array with regular interval or pitch, and the micro-needle structures 106 are identical to each other. The micro-needle structures 106 have a pyramid shape with a sharp tip, and the base attached to the planar electrode surface, such that the electrode 102 may be affixed to a skin surface (not shown) which may have different conditions, i.e. having sweat or grease on the skin surface, or having a rough morphology of the stratum corneum layer and/or the viable epidermis layer of the skin in contact with the electrode, or even roughen by a scar or body hair on the skin surface.

Alternatively, the micro-needle structures 106 may be provided in other shapes, such as cone shape or cylindrical shape, having a predetermined length protrude from the planar surface of the electrode to overcome the roughness of the skin surface. In addition, the electrode 102 is preferably flexible such that the electrode 102 may be flexibly applied on a skin surface of a patient with a curvature. Referring to FIG. 1B, the micro-needle electrode 102 may be bent into a curve using a pair of tweezers.

The micro-needle electrode 102, preferably, includes two layer of electrically conductive metals-gold (Au) 108 and nickel (Ni) 110, where the skin surface is in contact with the gold layer 108 when the micro-needle electrode 102 is applied on the skin surface. The gold layer 108 may be of a thickness of 500 nm to ensure the sheet resistance of the electrode is in an acceptable range for various applications, and the relatively thin layer of gold 108 is backed by another thin layer of nickel 110, preferably around 5 μm such that the micro-needle electrode 102 is mechanically strong enough for practical applications of the micro-needle electrode 102 while maintaining its flexibility. It should be appreciated by a skilled person in the art that the thickness of the gold layer 108 and nickel layer 110 may be varied in other designs which may require different electrical/mechanical properties, and it is also possible that the electrically conductive material may consist of other electrically conductive material, i.e. metallic or compound-based conductive material, or combinations of multiple layers of conductive material which may be used to form the necessary micro-needle structures 106 on a substantially planar sheet of conductive material using any suitable fabrication processes.

The photo in FIG. 1B highlights the ultra-high flexibility of the thin MNE 102 featuring ˜5.5 μm thickness, which offers seamless integration with the skin for high-fidelity signal detection. FIG. 1C and FIG. 1D show the tilted view and top view of the MNE 102 characterized by optical microscopy. Two metallic layers (Au/Ni) of conductive film are electrodeposited with uniform coverage and a smooth surface, which is suitable for wearable electrodes.

Referring back to FIG. 1A, there is shown a schematic diagram illustrating the method 100 in accordance with a preferred embodiment of the present invention. In this embodiment, the fabrication process generally includes a preparation of a stamp or a mold, such as using a composite material called OrmoStamp, fabricated using the nanoimprinting lithography, and the micro-needle electrode 102, i.e. the conductive layers of material with the micro-needle structures 106 formed across the planar metal sheets.

Preferably, the stamp is a negative stamp 104 for producing “positive structure”, i.e. voids 112 (the reverse image of the protruding structures) on the stamp would become protrusions (the micro-needle structures 106), on deposited layers after the deposited layers are released from the negative stamp 104. In the preferred embodiment as further described as follows, the negative stamp 104 is electrically conductive, and the at least one layer of electrically conductive material is deposited onto the negative stamp 104 by electrodepositing.

Preferably, to facilitate electrodepositing using the negative stamp 104, the negative stamp 104 is electrically conductive, and wherein the at least one layer of electrically conductive material is deposited onto the negative stamp 104 by electrodepositing. The layer of gold 108 and the layer of nickel 110 are sequentially electrodeposited onto the negative stamp 104 employing a step-up current source. A fabrication process for the preparation of the negative stamp is further described as follows.

Preferably, the negative stamp 104 includes a layer of indium tin oxide (ITO) 104A covering a stamp substrate 104B, where the stamp substrate 104B may be formed using a UV-curable molding material such as OrmoStamp. The OrmoStamp mold or the stamp substrate 104B may be pattered using a positive mold such as a PDMS mould 114 or a hard mold fabricated using lithography and etching techniques.

The process starts with the preparation of a conductive mold, i.e. the negative stamp 104 comprising the OrmoStamp substrate layer 104B and the ITO coating 104A, which are further structured with micro-needles having a pyramid shape (i.e. the voids 112). Referring to FIG. 1A, the preparation involves using a UV-curable molding material (OrmoStamp) to replicate micro-needle structures 106 from a PDMS mold 114. This nanoimprinting lithography step is crucial in creating a base that allows for the effective deposition of conductive materials in later stages. Next, a layer of ITO 104A, approximately 250 nm thick, is sputtered onto the OrmoStamp surface via magnetron sputtering, or other deposition process known in the field. This ITO layer serves to make the microneedle mold conductive and therefore suitable for the subsequent electrodeposition process.

The following stage involves the sequential electrodeposition of gold (Au) 108 and nickel (Ni) 110 with a thickness of ˜500 nm and ˜5 μm, respectively, onto the micro-needle ITO substrate by employing a step-up current source and strategy. An Au layer 108 is chosen for its superior biocompatibility, robust physical and chemical stability and high electrical conductivity, making it an ideal candidate for direct skin contact. Concurrently, a Ni layer 110 is electrodeposited on to the substrate or the negative stamp 104 to bolster the hardness and rigidity of the metallic film and mitigate manufacturing costs. A step-up current source is applied because an unduly high deposition current would result in an overly rapid electrodeposition rate, consequently leading to the formation of a coarse and non-dense metal film susceptible to defects like cracking or chipping on the ITO surface.

After the electrodepositions, the MNE 102 on the negative substrate 104 is cleaned with deionized (DI) water to remove any leftover electrolytes and is finally blown dry with nitrogen gas. As a result, a freestanding Au/Ni metal layer exhibiting the microneedle structure is obtained by carefully peeling off the electrodeposited metal layers from the ITO substrate. This step results in the final product, a flexible, highly conductive MNE 102. This entire process successfully addresses resolution limitations often encountered in current fabrication methods by fabricating microneedle electrodes with microscale features and offers a cost-effective solution for producing MNEs.

Unlike other fabrication methods, the method of the present invention eliminates the use of expensive equipment, and the ITO substrate can be reused for multiple electrodepositions, enabling cost-effective and facile procedures suitable for practical applications.

Other metals that can be electrodeposited and hold similar biocompatibility and mechanical properties can also be used. Electrodeposition is carried out using an electrochemical workstation (CHI 660E), which allows for precise control of the deposition process via a constant current. The electroplated sample is then thoroughly cleaned with deionized water to eliminate any remaining electrolytes. It is subsequently dried using nitrogen gas to prepare it for the final step.

With reference to FIGS. 2A to 2C, the microneedle structures 106 of the MNE 102 of with various key dimension produced by the method of FIG. 1A. With reference also to the microscopic images as shown in FIGS. 1B to 1D, it is observed that the metal layer is deposited with uniform coverage and a smooth surface, which is suitable for wearable electrodes. The base length or diameter, pitch, and height of the microneedles are ˜ 50, ˜70 and 34 μm, respectively, which follow the dimensions of the microstructured PDMS mold 114. Microneedles with other geometries (e.g. cone, cylinder, etc.) and dimensions (e.g. height from 20 to 200 μm, pitch from 50 to 500 μm, length from 20 to 300 μm, etc.) can also be used. In addition, referring to FIGS. 2A and 2B, the dimensions of the microneedle structures 106 on each of the individual layers, i.e. the nickel layer 110, the gold layer 108, the negative stamp 104 are all the same and replicating the microneedle structures on the original PDMS stamp 114 as shown in FIG. 1A.

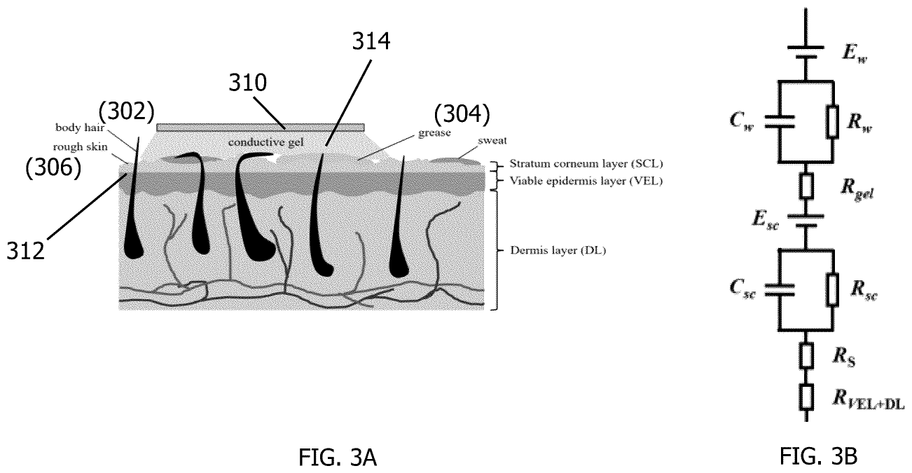

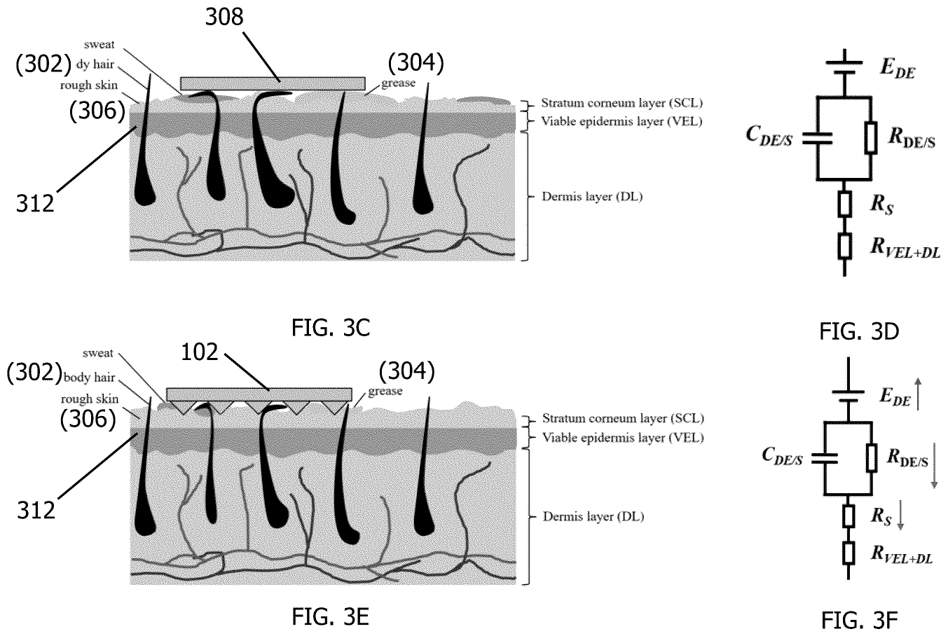

Now referring to FIGS. 3A to 3F, it is worth mentioning that hair 302, grease 304, and skin irregularities 306 can hinder optimal skin contact during bio-signal acquisition, potentially impacting detection accuracy. As depicted in FIGS. 3E to 3F, the MNE 102 of the present invention is applied on a skin surface 312 at accommodating body hair. These microneedles 106 effectively bypass sweat and grease and maintain good contact with the skin's uneven texture, providing superior conductivity compared to the flat electrodes 308. Additionally, the MNE 102 have a larger contact area due to their increased total surface area, thus the equivalent contact resistance is reduced, when compared to the equivalent circuit of the model with the planar electrode 308 as shown in FIGS. 3C and 3D. This contributes to decreased electrode-skin interface impedance (EII) and reduced motion artifacts during bio-signal measurements, compared also to the wet Ag/AgCl electrodes 310 and the equivalent circuit as shown in FIGS. 3A and 3B due to the additional resistance/impedance introduced by the conductive gel layer 314. Moreover, due to the improved fabrication resolution of the microneedle structures 106, a painless and more comfortable wearing experience is provided for the users without their noticing the existence of the microneedles on their skin.

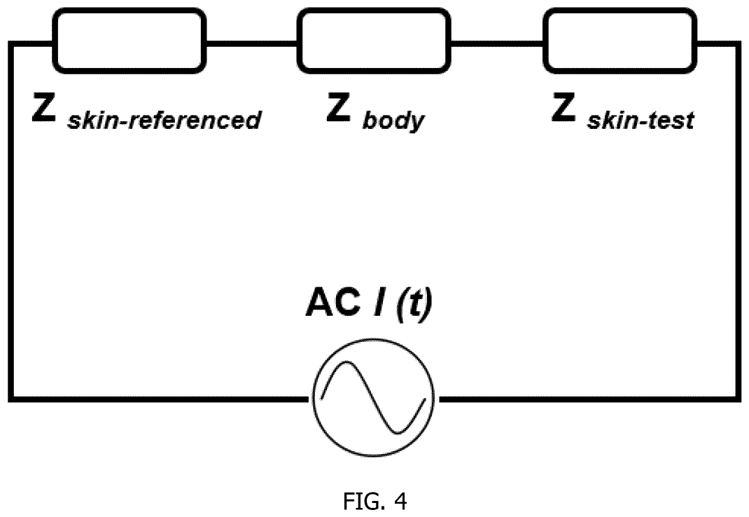

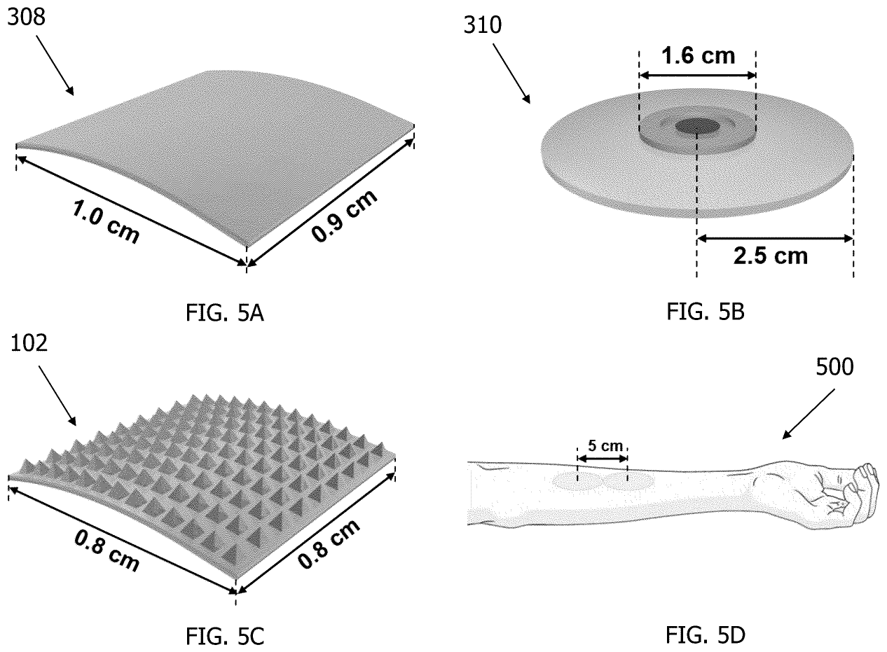

The EII is critical for the acquisition of high-quality electrophysiological signals. A lower EII indicates better signal quality with a higher SNR and reduced baseline drift. An EII analysis between electrodes and skin was conducted on the human forearm with a pair of electrodes placed on the inner side of the forearm using medical tape with a separation distance of 5 cm, with reference to FIGS. 4 to 5D). The analysis was based on the equivalent circuit model as shown in FIG. 4. FIG. 5A is schematic image of a planar electrode 308 (0.9 cm²), FIG. 5B is schematic image of a wet Ag/AgCl electrode 310 (2.01 cm²), FIG. 5C is schematic image of a MNE 102 (0.64 cm²) and FIG. 5D is an illustration of a patient wearing a pair of electrodes on his arm 500 for an EII test. In this example, a patch electrode for biopotential monitoring may comprising a flexible micro-needle electrode (MNE) as earlier described; and an electrical conductor arranged to electrically connect the flexible micro-needle electrode to a biopotential monitoring device, for later on analysis such as ECG, EMG, EEG or EOG.

In this example experiment, the EIIs analysis was conducted for three types of electrodes, including wet gel Ag/AgCl electrodes (Cathay Manufacturing Corp, CH55RB), MNEs and electroplated planar electrodes, respectively. To minimize the effect of different connections between electrodes and the instrument, the electrodes were affixed to standard snap connectors, where the gels were removed with the Ag/AgCl plate exposed. Considering contact areas between diverse types of electrodes and the skin, the EII values were normalized to represent the electrode performance more accurately in the following manner: |EII|_{normalized}=|EII|_{measured}×A, where |EII|_{measured} is the measured EII (kΩ), A is the contact area (cm²), |EII|_{normalized} is the normalized EII (kΩ*cm²).

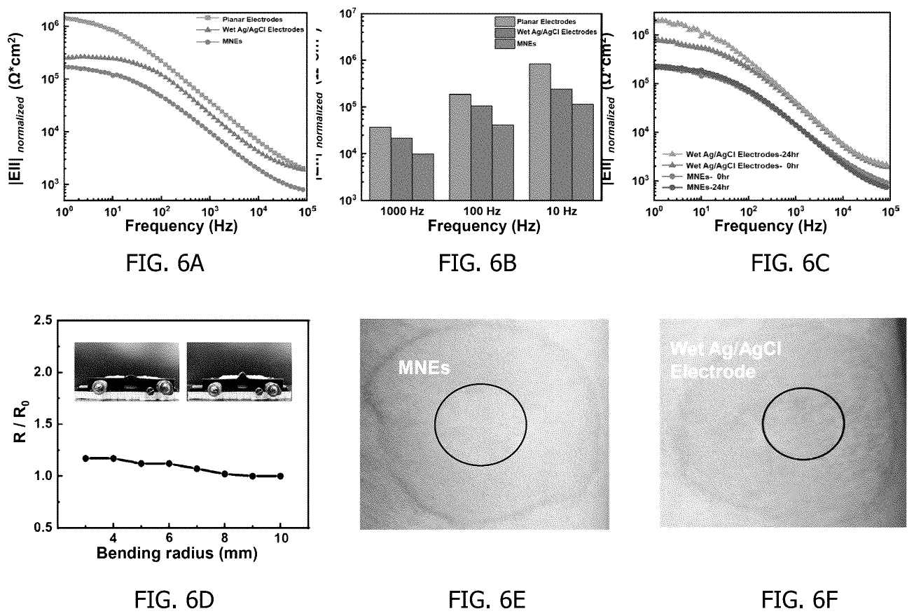

FIG. 6A exhibits the normalized EII spectra of three types of electrodes with the current frequencies ranging from 1 to 10⁵ Hz and the values of |EII|_{normalized} at specific frequencies were extracted and shown in FIG. 6B. Notably, the MNEs demonstrate reduced |EII|_{normalized}, surpassing the performance of both the wet electrodes and planar electrodes. This is particularly evident in the low-frequency bandwidth (<1 kHz), which encompasses the frequency content range of EMG and ECG signals. Specifically, the |EII|_{normalized} of MNEs is 4.7×10⁴ kΩ*cm² at 100 Hz, a decrease of nearly 5 and 3 times relative to planar electrodes and wet electrodes, respectively. The diminished EII of MNEs, as opposed to planar electrodes, strongly supports the significant contribution of microneedle structures in enhancing signal quality by increasing the effective skin contact area. In particular, due to the pyramidal geometry of the microneedles, which protrude from the flat surface, their four-sided structure presents a larger surface area compared to unstructured, flat electrodes.

Moreover, the raised profile of the pyramid allows it to traverse the skin's uneven surface, including wrinkles, and surface impurities such as sebum, sweat and hair, thereby facilitating direct skin contact. In contrast, flat electrodes may reduce skin contact due to gaps created by surface unevenness and impurities. By increasing the effective skin contact area, there is also a reduction in interference and the impact of surface impurities on electrophysiological signals detected by MNEs.

The long-term usability of MNEs was further evaluated by measuring the |EII|_{normalized} of MNEs and Ag/AgCl after 24 hours, and it is observed that the flexible micro-needle electrode may be worn for at least twenty-four hours without loss of performance. FIG. 6C shows that the EII of MNEs remains stable after 24 hours with a slight increase. In contrast, the |EII|_{normalized} of wet Ag/AgCl after 24 hours displays a significant increase, with almost three times the increment compared to EII measured immediately. The degradation of wet Ag/AgCl could be explained by the gel drying out over time, i.e., the observed phenomenon can be attributed to the degradation and drying of conductive gel in wet Ag/AgCl electrodes. Over time, these changes result in increased contact resistance of the wet Ag/AgCl electrodes. Contrarily, the design and structure of MNEs avoid such issues, making MNEs a more favorable selection for long-term physiological signal acquisition.

Next, the electro-mechanical stability of MNEs was examined. The MNEs were affixed to a poly (ethylene terephthalate) (PET) substrate and then subjected to bending at varying angles. The 4-probe resistance change was monitored. A negligible change in resistance was observed as the electrode was bent at a bending radius ranging from 3 to 10 mm as shown in FIG. 6D, suggesting that it is capable of working as a flexible electrode. The strengthened flexibility of MNEs could be related to the microneedles, which act as a stress reservoir to accommodate the strain energy imparted from the external force.

The inventors devised that another essential requirement for wearable electrodes is good comfort and high biocompatibility. Benefiting from the small features of the microneedle arrays on MNEs, painless and minimally invasive electrophysiological detection was achieved, which is suitable for long-term monitoring. With reference to FIGS. 6E and 6F, the skin condition after wearing the MNEs and the wet Ag/AgCl electrodes for 2 hours were compared. The regions within the circles represent the areas where the two types of electrodes contacted the skin, while the surrounding regions represent the coverage of the same medical tape. It is clearly shown that the MNEs, without the utilization of conductive gel, can largely eliminate the red print compared with the wet Ag/AgCl electrodes. No skin irritation was reported in the sensing area while more serious redness was observed in the tape-covered area.

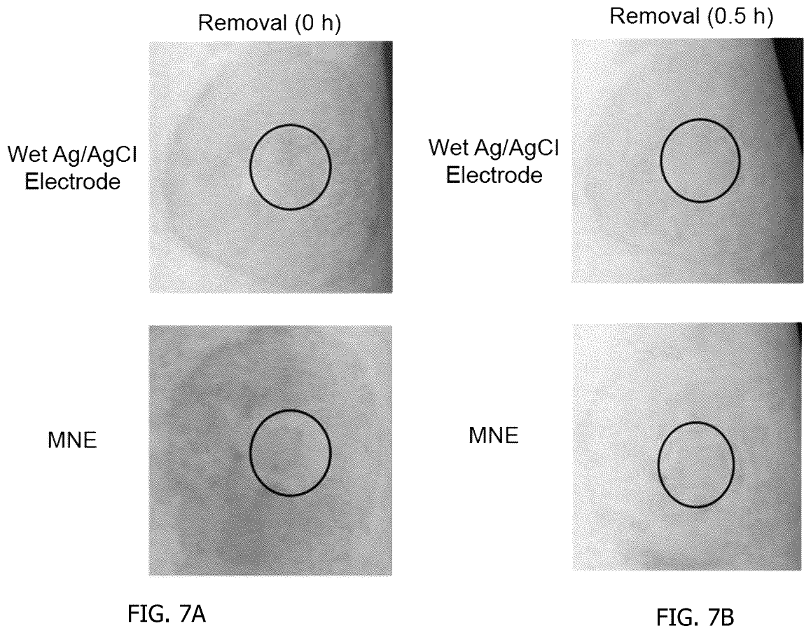

With reference to FIGS. 7A and 7B, it is observed that the marks of the MNEs can disappear entirely within 0.5 hours after removal. Conversely, the residual redness from the gel electrode were still visible even after the same duration. Beyond this, compared to uncomfortable invasive microneedles, non-invasive microneedles do not leave behind micro-perforations and surface material residue on the skin surface during the process of insertion and retraction. This evidences the excellent biocompatibility and biosafety of the non-invasive MNEs of the present invention during prolonged wear.

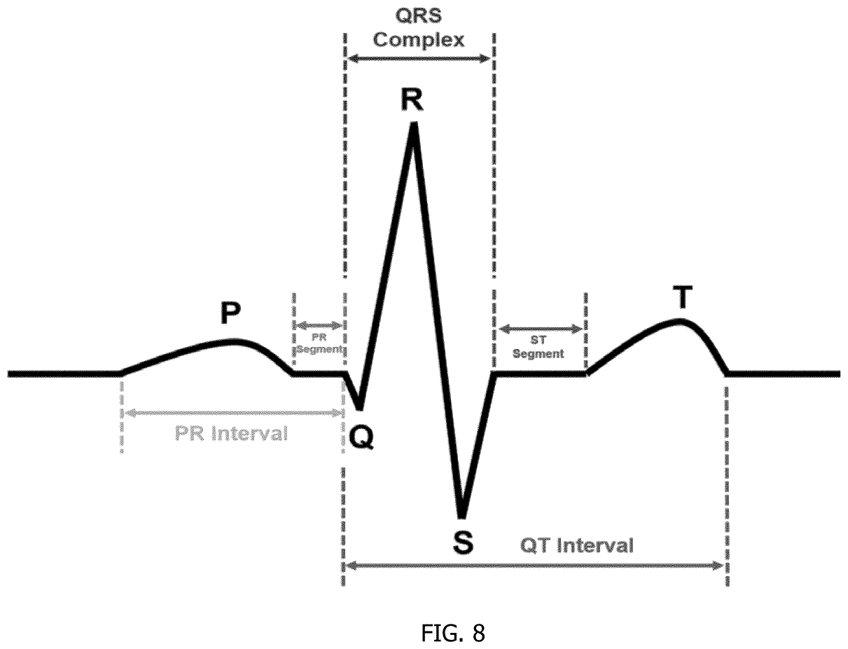

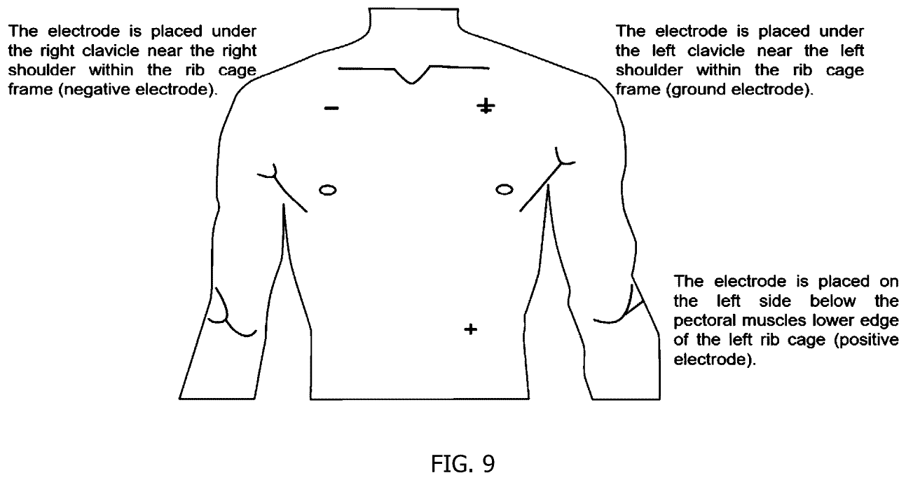

The MNEs of the present invention can be used as wearable dry electrodes to detect epidermal biopotentials. Within the human body, every cyclical contraction of the heart generates a periodic electrical signal change across various body regions, a phenomenon denoted as ECG, is illustrated in FIG. 8. For example, referring to FIG. 9, to record the ECG signal, two MNEs were positioned beneath the right clavicle and below the left clavicle, while a wet Ag/AgCl electrode, acting as a reference, was placed under the left rib cage.

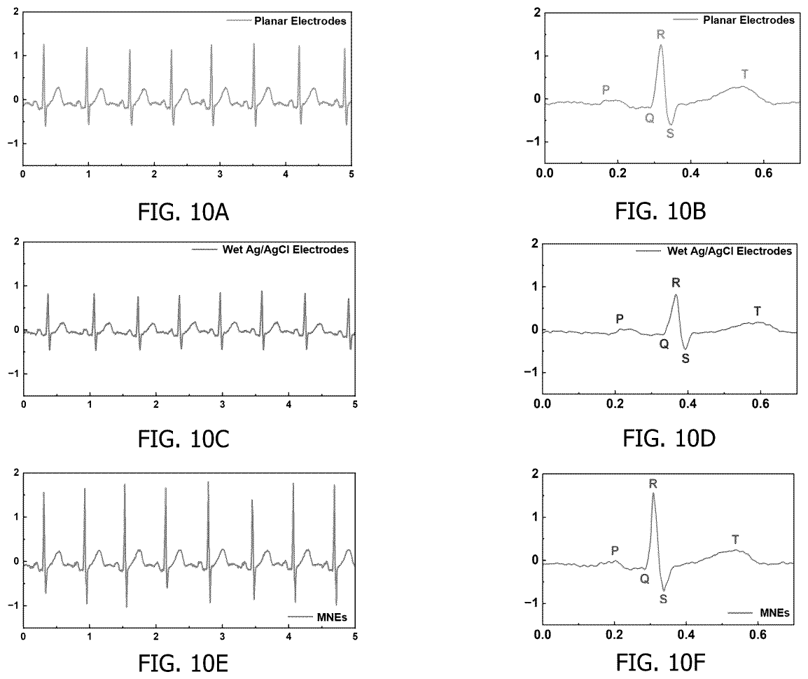

Wet Ag/AgCl and electroplated planar electrodes were also utilized as working electrodes to measure ECG signals for comparison. FIGS. 10A, 10C and 10E display the ECG signals recorded by the three types of electrodes at the same test spots during static states. For a detailed comparison, zoomed-in figures for a single characteristic wave from all electrodes are shown in FIGS. 10B, 10D and 10F. Notably, the signals acquired from the MNEs demonstrate a larger peak-to-peak voltage (2.28 mV) with a more distinct ECG characteristic wave compared to the wet electrodes (1.28 mV) and planar electrodes (1.85 mV). The larger amplitude and distinguishable ECG characteristic waves captured by MNEs are readily discernible, showing the promise for clinical diagnosis of various cardiac signal abnormalities.

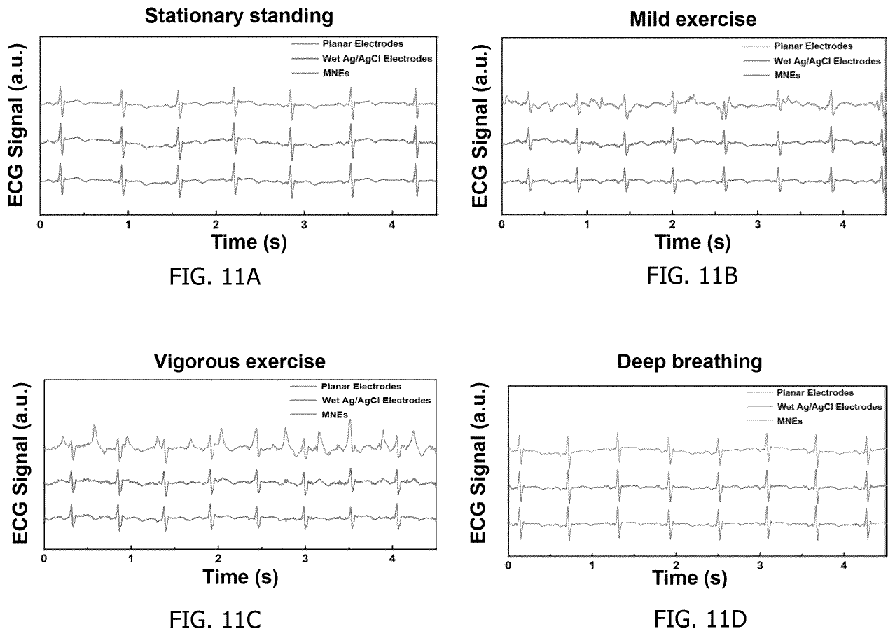

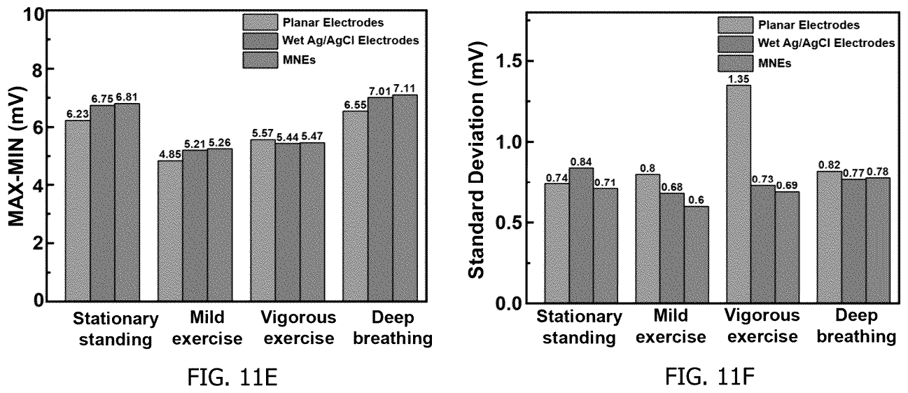

In addition, in the example experiments, ECG signals were also captured during various body movements to validate the anti-artifact capabilities of the MNEs. The ECG signals were also collected by three types of electrodes including MNEs, flat electrodes, and wet Ag/AgCl electrodes simultaneously. Four kinds of continuous active states, including resting (static standing), light exercise (slow walking in place), vigorous exercise (running in place), and deep breathing were performed by a volunteer (24-year-old male). Signals from all three electrodes were recorded for 60 seconds and selected for 4.5 seconds at the same time during each activity state as displayed in FIGS. 11A to 11D.

Referring to FIG. 11A, the ECG signals recorded by the three electrodes displayed comparable performances with distinguishable PQRST waveforms in the static standing state. However, a noticeable shift in the baseline of the ECG signals was observed for the planar electrodes when the volunteer engaged in slow walking as shown in FIG. 11B. The baseline fluctuations worsen during more vigorous exercise, such as running in place as shown in FIG. 11C. Due to the adhesive ion gel, the wet Ag/AgCl electrodes exhibited better resistance against motion artifacts than the flat electrodes during body movement.

Advantageously, the MNEs displayed the best anti-artifact performance among the three types of electrodes, maintaining a stable baseline even during intense movement. Finally, upon transitioning from vigorous exercise to deep breathing, as shown in FIG. 11D, the ECG signals recorded by all three types of electrodes could be clearly distinguished without any baseline drift. This observation demonstrates that the differences in ECG signals during mild exercise and vigorous exercise, as expounded previously, are attributed to the anti-artifact capabilities of the electrodes, thereby excluding other interfering factors. Moreover, the difference between the maximum and minimum peak values (MAX-MIN) and the standard deviation (SD) of the QRS complex were computed to evaluate the artifact levels in the ECG signals, with reference to FIGS. 11E and 11F. The MNEs exhibited the highest MAX-MIN of the QRS complex with the slowest SD during the active states. In summary, the measurements reveal the MNEs' advanced proficiency to suppress motion artifacts, surpassing both the planar and wet electrodes. This enhanced capability of MNEs stems from the protruding pyramid-shaped microneedles, offering not only an enhanced effective contact area but also delivering transverse tangential forces to counter motion.

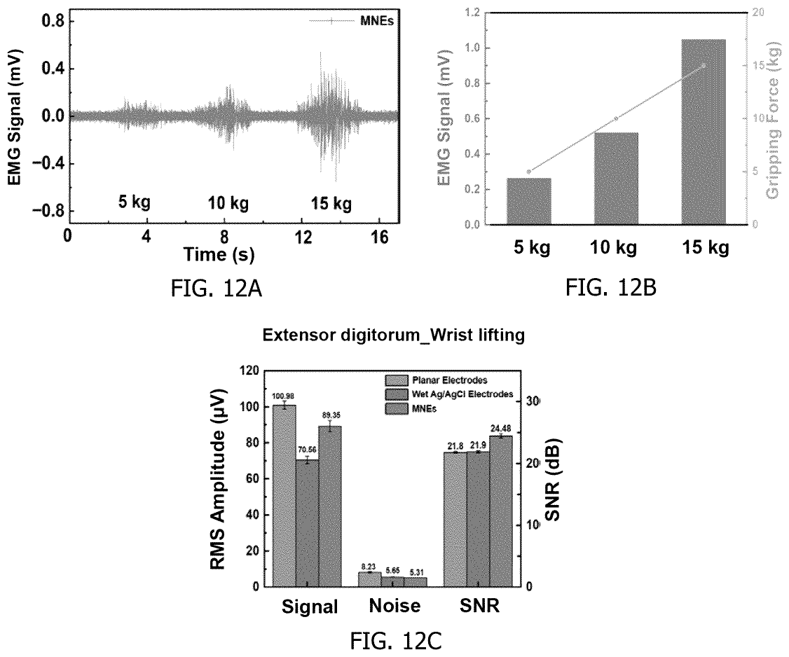

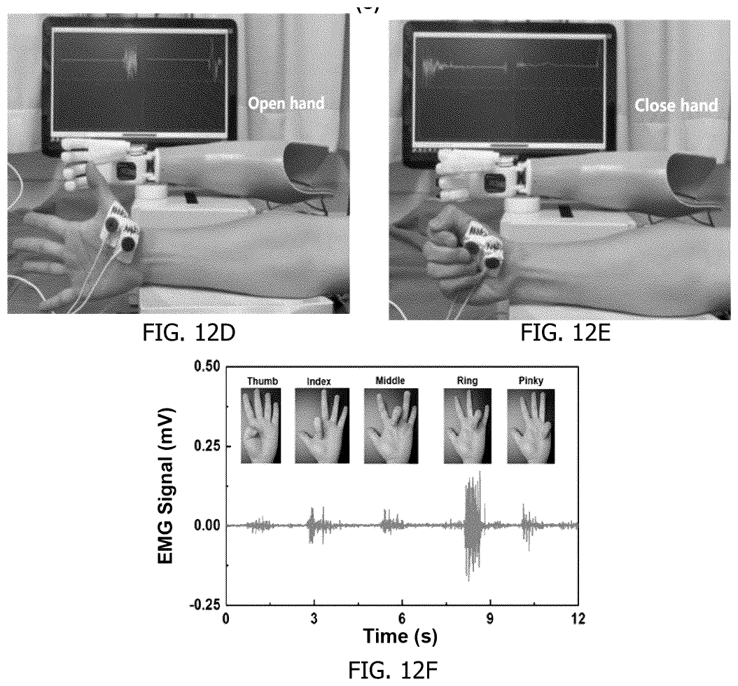

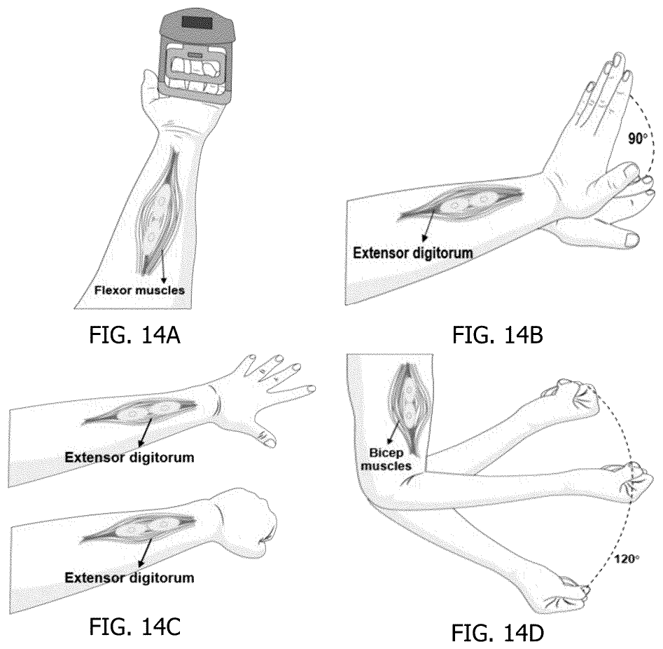

In addition, the MNEs fabricated in accordance with embodiments of the present invention can further be used as dry electrodes for EMG tests that detect the action potentials produced by muscle contractions. EMG signals play a critical role in various applications such as medical diagnosis, athletic training, human-robot interactions, etc. With reference to FIGS. 12A to 12F, the capability of the MNEs in EMG signal detection was evaluated. The surface EMG recordings were first taken by placing a pair of MNEs parallel and laterally along the flexor digitorum muscle on the inner side of a volunteer's forearm as shown in FIG. 14A, with a reference electrode placed on the ankle.

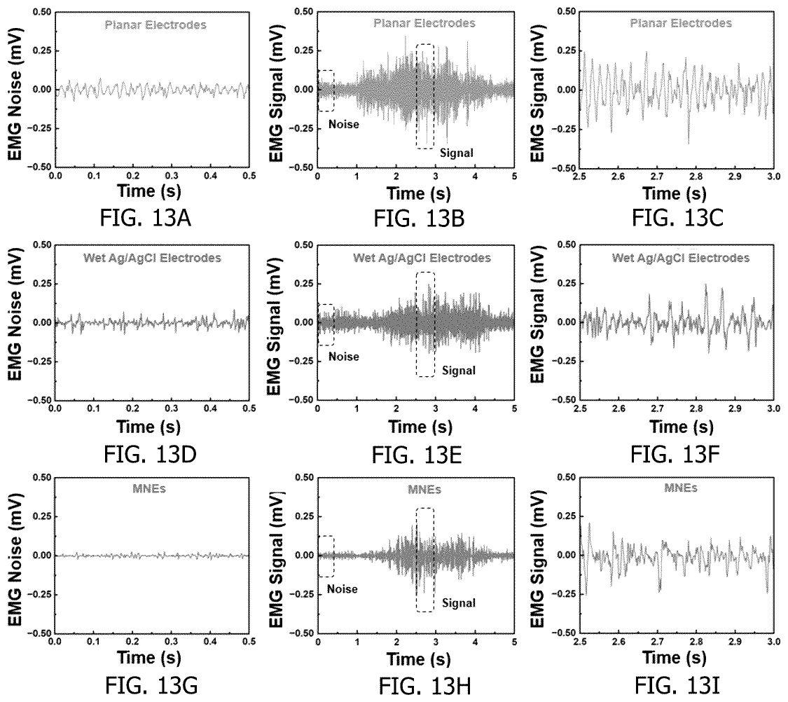

In the experiment, the volunteer held a grip dynamometer to measure the gripping force using the same hand applied with the electrodes. In order to comparatively assess the detection capacities of different electrodes for EMG signals, participants were asked to repeatedly exert a handgrip dynamometer up to 5 kg without prior knowledge of the electrode type. Between each exertion, sufficient rest was ensured for the muscles, and consistency in electrode placement was maintained. FIGS. 13B, 13E and 13H depict the EMG signals generated from the contraction of flexor muscles captured by three types of electrodes. They all exhibited comparable peaks and valleys corresponding to motion activation and relaxation. To further evaluate the quality of the detected EMG signal, 500-ms data from the center of the high-amplitude EMG signal produced by muscle activation were extracted, referring to FIGS. 13C, 13F and 13I, while the noise segments were obtained by selecting 500-ms data from the center of the relaxing part between each two EMG signals FIGS. 13A, 13D and 13G. Serving as EMG detection electrodes, the MNEs exhibited lower baseline noise and a more distinguishable signal in comparison to the other two types of electrodes, thereby facilitating the interpretation of muscle activity.

To further assess the capability of the MNEs, EMG signals at different gripping forces were measured using MNEs as shown in FIG. 12A. The peak-to-peak amplitude and the signal intensity are consistent with the gripping force as shown in FIG. 12B, which is indicative of the gripping force. To realize wearable applications, dry electrodes, which are able to maintain reliable performance during body movement, are highly desirable. Therefore, EMG detections during body movements were conducted with the electrodes attached to the noticeably curved skin surfaces under different scenarios as shown in FIGS. 14A to 14D.

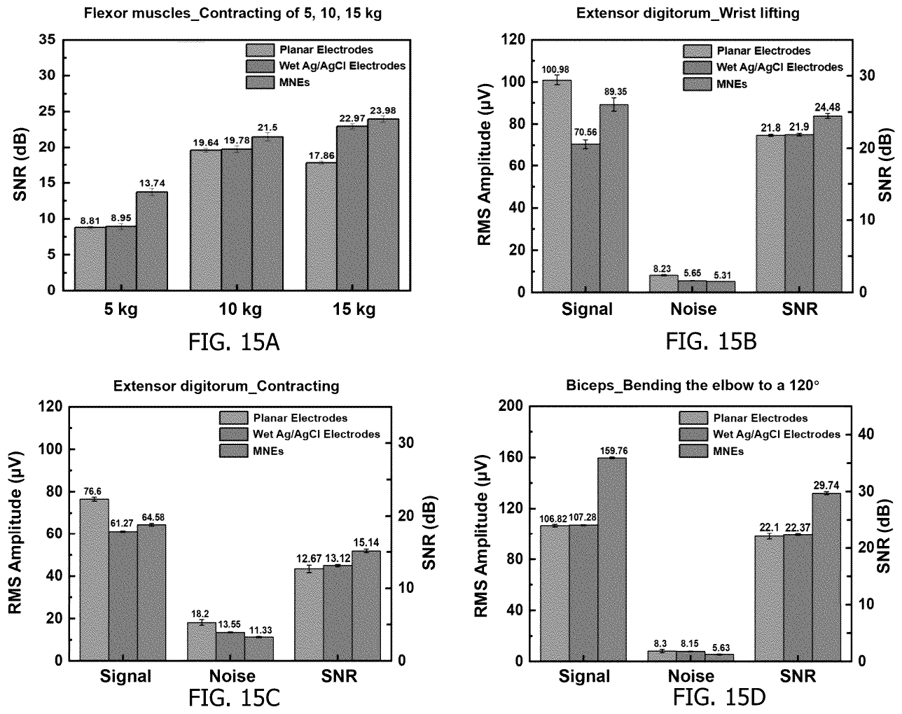

With reference to FIG. 12C, despite the high intensity of muscle activities during wrist lifting as shown in FIG. 14B, the MNEs exhibited the lowest RMS values for baseline noise in comparison to the other two types of electrodes across various movements. Additionally, EMG detections during different body movements, including contraction as shown in FIG. 14C, and elbow bending as shown in FIG. 14D were conducted and displayed in FIG. 12C and FIGS. 15A to 15D. Among the three electrodes assessed, the MNEs demonstrated significantly lower noise levels and superior SNR compared to both the wet Ag/AgCl electrodes and flat electrodes. Although the flat electrodes procured a higher RMS per muscle contraction, they suffered from comparatively high noise, resulting in lower SNR than the MNEs. These findings confirm that MNEs offer superior electro-mechanical stability during body motion, which is advantageous for electrophysiological monitoring in epidermal electronics.

The accurate and imperceptible electrophysiological detection competence of MNEs shows great potential for controlling robotic hands with high precision. The EMG signal derived from hand opening and closing movements as shown in FIG. 14C, captured by the MNEs, can function as a human-machine interface to orchestrate the real-time opening and closing actions of a robotic hand, as demonstrated in FIGS. 12D and 12E. Additionally, the MNEs can also identify the low-amplitude EMG signals originating from a finger executing flexion or extension. As shown in FIG. 12F, the distinguishable EMG signal intensities measured by the MNEs, which showcases the potential of MNEs in human-machine interface applications. Overall, it can be envisioned that the MNEs of the present invention, being highly conductive, conformable, and mechanically flexible, are promising for more complex applications involving human-robot interactions.

The materials used to fabricate the MNEs of the present invention include a polydimethylsiloxane (PDMS) microneedle mold, with a periodicity of ˜70 μm, a basal length of ˜50×50 μm, and a height of ˜34 μm. It was obtained from Nanjing University. The OrmoPrime08 and OrmoStamp materials were purchased from Micro Resist Technology GmbH. The electroplating solution, including Au (Plug N′ Plate Gold Solution), and Ni (Plug N′ Plate Nickel Solution), were procured from CASWELL, USA. The wet Ag/AgCl electrodes (CH55RB) were bought from the Cathay Manufacturing Corp.

The fabrication of the OrmoStamp Mold started with a layer of OrmoPrime (˜130 nm film thickness) that was first spin-coated onto a 2 cm×2 cm clean glass at 4000 rpm for 60 s and then baked on a hotplate at 150° C. for 5 min. After the glass was cooled down to room temperature in the air, a UV-curable molding material (OrmoStamp) was cast and subsequently solidified to accurately replicate the microstructure from the PDMS microneedle mold by exposing it to 365 nm UV light using a photolithography machine (URE 2000/35, Chinese Academy of Sciences, China) for 300 s. Subsequently, a layer of ITO with a thickness of ˜250 nm was deposited onto the OrmoStamp surface via magnetron sputtering to render the microneedle mold conductive for electrodeposition.

The electrodeposition of Ni and Au was achieved with a step-up current source using an electrochemical workstation (CHI660E). The ITO mold was sequentially immersed in the Au and Ni electroplating solution for the electrodeposition of Au and Ni while connected to the current source cathode. Correspondingly, the anode of the current source was linked to platinum (Pt) and Ni wands dipped in the solution in sequence. Upon completion of electroplating, the sample was thoroughly cleansed with deionized (DI) water to eliminate any residual electrolytes and was finally dried in nitrogen gas. Then a freestanding Au/Ni metallic layer exhibiting microneedle structure was obtained through a cautious peeling-off process from the ITO substrate.

To evaluate the ability of the MNEs to monitor epidermal biopotential, the EII was measured by a two-electrode system using electrochemical workstation (CHI660E). Before the tests commenced, the skin area where the electrodes would be placed was thoroughly cleaned with a 75% alcohol solution. To minimize the impact of electrode-device connections in the study, both the MNEs and planar electrodes were adhered to an Ag/AgCl plate after removing the gel, using a conductive adhesive. Subsequently, they were linked to the instrument using standard button connectors. The center-to-center distance between the two electrodes was precisely set as 5 cm.

For ECG Measurements a volunteer (24-year-old male) was required to wear two testing electrodes, one placed below his right clavicle and the other beneath his left clavicle. Additionally, a wet Ag/AgCl electrode served as a reference and was positioned under his left rib cage as shown in FIG. 9. The ECG signals during static states (an upright seated posture) were first recorded and analysed by the data acquisition system (PowerLab 26T, ADInstruments Pty Ltd), with the bandpass filter set between 0.5-50 Hz. A waterproof, stretchable medical adhesive (3M Tegaderm) was employed to encapsulate the connection point to secure and stabilize the recording systems. Afterward, the subject sequentially engaged in five distinct active states, namely, static states (an upright seated posture), stationary standing, mild exercise (slow walking in place), vigorous exercise (running in place), and deep breathing. The signals during these activities were recorded by three different types of electrodes simultaneously using the customized 32-channel bio-signal recording system from the Neural Engineering & Clinical Electrophysiology Laboratory at the University of Hong Kong. The filtering parameters remained consistent during the measurements.

The EMG signals were also measured and analysed by the data acquisition system (PowerLab 26T, ADInstruments Pty Ltd), utilizing the same electrodes connection procedure as the ECG test. The surface EMG signals produced by the extensor digitorum, flexor digitorum, and biceps muscles were tested, with their locations depicted schematically in FIGS. 14A to 14D. The pair of electrodes was positioned with a center-to-center distance of 50 mm, and a reference electrode was specifically positioned at the ankle. The recorded EMG raw data, exhibiting low-frequency motion artifacts, underwent filtration processes to derive oscillation-free baselines imperative for signal-to-noise ratio (SNR) analyses. This was accomplished via the application of a two-part filter, inclusive of a 10 Hz high-pass finite impulse response (FIR) and a 50 Hz low-pass digital filter. Concurrently, power frequency disturbances were eliminated by deploying a 50 Hz harmonic infinite impulse response (IIR) comb digital filter.

Thus, the basic functions and performances of the MNEs were tested demonstrating that they can clearly monitor and transmit bio-signals, such as ECG and EMG. These MNEs feature microneedle dimensions down to tens of micrometers which address the shortcomings of commonly used wet Ag/AgCl electrodes and planar electrodes. The MNEs are fabricated with a cost-effective procedure involving magnetron evaporation of ITO on a microneedle mold and electrodeposition of microneedle metal layers. The fabricated MNEs show superior advances in electro-mechanical stability, biocompatibility, and comfort. The introduction of microstructures strengthens accurate detection capability for capturing electrophysiological signals like EMG and ECG, and has minimal motion artifacts that surpass that of the wet electrodes and planar electrodes. With regard to comfort, traditional gel electrodes necessitate skin pre-treatment and can cause discomfort and potential skin irritation. The MNEs, due to their micro-needle structures, can adhere comfortably to the skin without causing pain or irritation because the MNEs feature a unique microneedle structure with a few tens of micrometers.

The inventors devised that to ensure long-term biopotential monitoring with high-quality signals and minimal motion disruptions, the dry electrodes that can adhere exceptionally well to the skin while maintaining robust electro-mechanical stability and detection accuracy. Advantageously, the invention presents a novel, cost-effective, and scalable solution to surface biopotential monitoring by introducing highly conductive, flexible and ultra-thin microneedle electrodes (MNEs), fabricated using a 3D electrodeposition method. Unlike conventional Ag/AgCl gel electrodes, these MNEs eliminate the need for skin pre-treatment, discomfort, potential skin irritation, and the issue of signal degradation over time.

Compared to existing dry electrodes, the MNEs fabricated in accordance with embodiments of the present invention overcomes the problems of high materials cost, low adhesiveness, and low flexibility. This innovative design ensures that the MNEs have excellent conductivity, adhere comfortably to the skin without causing pain, and maintain a high signal-to-noise ratio, marking a significant advancement in the field of wearable electrophysiology monitoring.

Advantageously, the invention of MNEs leverage a cost-effective and scalable fabrication process based on 3D electrodeposition. The invented MNEs and the fabrication process solve long-standing challenges of existing electrodes used in surface biopotential monitoring. The MNEs of the present invention are dry electrodes that overcome signal degradation caused by gel evaporation in mostly used gel electrodes, and more importantly, improve the user comfort by reducing skin irritation. The microneedle structure on the metal film offers superior stability and therefore a high signal-to-noise ratio because of the anchoring capability of the microneedles slightly penetrating the skin surface. Moreover, the scalable and cost-effective manufacturing process of the present invention based on 3D electrodeposition makes it possible to produce such MNEs with consistent performance and significantly reduced cost.

The MNEs of the present invention have good conductivity and excellent electro-mechanical stability for wearable electrophysiology monitoring, which was confirmed by electro-mechanical bending tests. Featuring a microneedle height of ˜34 μm, the MNEs are capable of adhering to the skins without causing any pain, ensuring good wearability and electrode-skin contact. Furthermore, the MNEs demonstrated outstanding proficiency in detecting electrophysiological signals, boasting a 1.35 times greater signal-to-noise ratio (SNR) than planar electrodes and a 1.33 times enhanced SNR compared to wet electrodes under identical conditions.

In addition, the MNEs of the present invention also possess lower EII compared to the electroplated planar electrode and wet Ag/AgCl electrode, which is attributed to the increased overall electrode surface area, conformal contact with skin and the ability to circumvent obstructions caused by sweat and grease on the skin. Additionally, the accurate and reliable detection capability of MNEs for electrophysiological signals, including EMG and ECG, has been demonstrated with SNR higher than that of the planar and wet electrodes. Moreover, the MNEs are less susceptible to body movement, which is critical for electrophysiological monitoring. To fully exploit the potential applications of MNEs, human-robot interaction based on the EMG signals measured by MNEs was conducted. Taken together the MNEs emerge as an ideal dry electrode to replace the wet Ag/AgCl electrodes for long-term healthcare monitoring and human-robot interaction.

Further, the MNEs fabricated in accordance with embodiments of the present invention have demonstrated exceptional flexibility with almost no resistance variations when they are bent at the radius from 3 to 10 mm. In addition, conventional methods of fabricating electrodes often involve expensive equipment and procedures. The present invention overcomes these issues by leveraging 3D electrodeposition of conductive metals on a structured ITO substrate. This fabrication method of MNEs is scalable and cost-effective, including reuse of the ITO substrate mold for multiple electrodepositions and avoiding the use of expensive tools.

The above are only specific implementations of the invention and are not intended to limit the scope of protection of the invention. Any modifications or substitutes apparent to those skilled in the art shall fall within the scope of protection of the invention. Therefore, the protected scope of the invention shall be subject to the scope of protection of the claims.

1. A method for constructing a flexible micro-needle electrode (MNE) for biopotential monitoring comprising the steps of:
providing a negative stamp that has been structured with a plurality of micro-needle structures;
depositing at least one layer of electrically conductive material onto the negative stamp; and
peeling off the at least one layer of electrically conductive material from the negative stamp to obtain the flexible micro-needle electrode comprising the at least one layer of electrically conductive material defined with the plurality of micro-needle structures.

2. The method of claim 1, wherein the negative stamp comprises a stamp substrate fabricated using nanoimprinting lithography.

3. The method of claim 2, wherein the negative stamp is defined with the plurality of micro-needle structures having a pyramid shape, a cone shape or a cylinder shape.

4. The method of claim 2, wherein the plurality of micro-needle structures include a height in a range from 20 to 200 μm, a pitch in a range from 50 to 500 μm, and a length in a range from 20 to 300 μm.

5. The method of claim 2, wherein the step of providing the negative stamp that has been structured with a plurality of micro-needle structures comprises the steps of:
providing a molding material to replicate the plurality of micro-needle structures from a positive mold;
curing the molding material to provide the stamp substrate.

6. The method of claim 5, wherein the positive mold includes a positive PDMS mold defined with the plurality of micro-needle structures.

7. The method of claim 5, wherein the molding material includes a polymer molding material.

8. The method of claim 5, wherein the molding material is UV-curable.

9. The method of claim 2, wherein the negative stamp is electrically conductive, and wherein the at least one layer of electrically conductive material is deposited onto the negative stamp by electrodepositing.

10. The method of claim 9, wherein the negative stamp includes a layer of indium tin oxide (ITO) covering the stamp substrate.

11. The method of claim 10, wherein the step of providing the negative stamp that has been structured with the plurality of micro-needle structures further comprises the step of:
depositing a layer of ITO onto the stamp substrate to provide the negative stamp.

12. The method of claim 11, wherein the layer of ITO is deposited on the stamp substrate by sputtering.

13. The method of claim 10, wherein the layer of ITO is approximately 250 nm thick.

14. The method of claim 9, wherein the at least one layer of electrically conductive material includes gold (Au) and nickel (Ni).

15. The method of claim 14, wherein a layer of gold and a layer of nickel are sequentially electrodeposited onto the negative stamp employing a step-up current source.

16. The method of claim 14, wherein the layer of gold and the layer of nickel include respectively a thickness of 500 nm and 5 μm.

17. A flexible micro-needle electrode (MNE) for biopotential monitoring, comprising at least one layer of electrically conductive material defined with the plurality of micro-needle structures produced using the method in accordance with claim 11.

18. A patch electrode for biopotential monitoring, comprising a flexible micro-needle electrode (MNE) for biopotential monitoring in accordance with claim 17; and an electrical conductor arranged to electrically connect the flexible micro-needle electrode to a biopotential monitoring device.

19. The patch electrode in accordance with claim 18, wherein the is a dry electrode adapted to be worn by a patient.

20. The patch electrode of claim 19, wherein the flexible micro-needle electrode is adapted to be worn for at least twenty-four hours without loss of performance.
